(19) United States
(12) Patent Application Publication
(10) Pub. No.: US 2023/0020353 A1
(43) Pub. Date: Jan. 19, 2023

(54) SYSTEMS AND METHODS FOR OPTIMIZING A RISK ASSESSMENT PROCESS

(71) Applicants: Walter Lautz, San Tan Valley, AZ (US); Warren Lautz, San Tan Valley, AZ (US)

(72) Inventors: Walter Lautz, San Tan Valley, AZ (US); Warren Lautz, San Tan Valley, AZ (US)

(21) Appl. No.: 17/865,174

(22) Filed: Jul. 14, 2022

Related U.S. Application Data
(60) Provisional application No. 63/221,828, filed on Jul. 14, 2021.

(51) Int. Cl. G16H 50/30; G16H 10/20

(52) U.S. Cl. CPC G16H 50/30; G16H 10/20

(57) ABSTRACT

Systems and methods for optimizing a risk assessment process according to various aspects of the present technology may include a server, a display, and a user device including a graphical user interface, a risk assessment engine, and a database. The graphical user interface may be configured to receive user data from a user, wherein the user data may include identifying information. The risk assessment engine may be configured to compute a risk score and generate a risk profile utilizing the identifying information and the risk score. The risk profile may indicate whether the user is likely to be harmed. The database may be configured to store the risk profile. The server may be communicatively linked to the database over a communication network, wherein the server may be provided with selective access to the risk profile. The display may be configured to present the risk profile.

### CROSS REFERENCE TO RELATED APPLICATIONS

This application claims the benefit of U.S. Provisional Patent Application Ser. No. 63/221,828, filed on Jul. 14, 2021, and incorporates the disclosure of the application in its entirety by reference.

### BACKGROUND OF THE TECHNOLOGY

### State of the Art

Various law enforcement agencies require law enforcement agents to collect critical domestic violence, transient, mental health, and substance abuse information and then report the information to government and/or victim advocacy groups. For example, in a domestic violence incident between an abuser and a victim, performing a risk assessment and then reporting information associated with the risk assessment to government and/or victim advocacy groups is generally considered to be an important part in developing a suitable safety plan for the victim.

Conventional methods for performing a risk assessment generally involve asking the victim a series of non-standardized questions. In addition, conventional methods for sharing the risk assessment with government and/or victim advocacy agencies may be a complicated and time-consuming process requiring a significant amount of research and calculation to organize and present the risk assessment to the agencies.

Conventional methods for victim safety planning have not sufficiently addressed the need to simplify the process of sharing critical domestic violence, transient, mental health, and substance abuse information with government and/or victim advocacy groups that are best suited to keep the victim/respondent safe.

### SUMMARY OF THE TECHNOLOGY

Systems and methods for optimizing a risk assessment process according to various aspects of the present technology may comprise a server, a display, and a user device including a graphical user interface, a risk assessment engine, and a database. The graphical user interface may be configured to receive user data from a user, wherein the user data may comprise identifying information. The risk assessment engine may be configured to compute a risk score and generate a risk profile utilizing the identifying information and the risk score. The risk profile may indicate whether the user is likely to be harmed. The database may be configured to store the risk profile. The server may be communicatively linked to the database over a communication network, wherein the server may be provided with selective access to the risk profile. The display may be configured to present the risk profile.

### BRIEF DESCRIPTION OF THE DRAWINGS

A more complete understanding of the present technology may be derived by referring to the detailed description when considered in connection with the following illustrative figures. In the following figures, like reference numbers refer to similar elements and steps throughout the figures.

FIG. 1 is a block diagram of a system in accordance with an exemplary embodiment of the present technology;

FIG. 2 representatively illustrates a flow diagram for operating the system in accordance with a first embodiment of the present technology; and

FIG. 3 representatively illustrates a flow diagram for operating the system in accordance with a second embodiment of the present technology.

### DETAILED DESCRIPTION OF EXEMPLARY EMBODIMENTS

The present technology may be described in terms of functional block components. Such functional blocks may be realized by any number of components configured to perform the specified functions and achieve the various results. For example, the present technology may employ various communication networks, databases, displays, graphical user interfaces, risk assessment engines, and servers, which may carry out a variety of functions.

The present technology may be practiced in conjunction with any number of systems and methods for providing a computer-implemented system or method for optimizing a risk assessment process. Further, the present technology may employ any number of conventional techniques for receiving, storing, transmitting, and displaying data.

The particular implementations shown and described are illustrative of the technology and its best mode and are not intended to limit the scope of the present technology in any way. Indeed, for the sake of brevity, conventional manufacturing, connection, preparation, and other functional aspects of the system may not be described in detail. Furthermore, the connecting lines shown in the various figures are intended to represent exemplary functional relationships and/or steps between the various elements. Many alternative or additional functional relationships or physical connections may be present in a practical system.

Various representative algorithms may be implemented with any suitable combination of data structures, objects, processes, routines and/or other programming elements. Software and/or software elements according to various aspects of the present technology may be implemented with any suitable programming or scripting language, such as C, C++, Java, COBOL, assembler, PERL, HTML, PHP, and the like. Further, the present technology may employ any number of conventional techniques for data transmission, signaling, data processing, network control, and the like.

According to various aspects of the present technology, a user may interact with the system by any input device such as a keyboard, a mouse, a handheld computer, a cellular phone such as a Smartphone that may have access to the Internet, and the like. Similarly, the present technology may be used in conjunction with any type of personal computer, network computer, workstation, minicomputer, or the like running any operating system such as any version of Windows, MacOS, or any other operating system, whether now known or known hereafter in the art. Moreover, the present technology may be implemented with TCP/IP communications, IPX, OSI or any number of existing or future protocols.

The present technology may be embodied as a method, a system, a device, and/or a computer program product. Accordingly, the present technology may take the form of a software embodiment, a hardware embodiment, or an embodiment combining aspects of both software and hardware. Furthermore, the present technology may take the form of a computer program product on a computer-readable storage medium having computer-readable program code embodied in the storage medium.

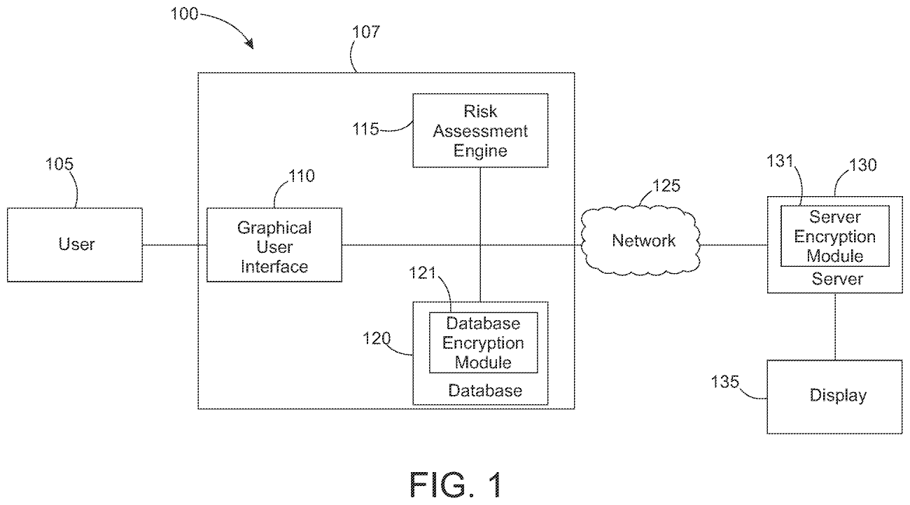

Referring to FIGS. 1-3, systems and methods for optimizing a risk assessment process according to various aspects of the present technology may be representatively illustrated by a system 100 for access by a user 105. The user 105 may be any suitable person, such as a victim of a domestic violence incident or someone experiencing homelessness, mental health, and/or substance abuse issues, a law enforcement agent, a victim advocate, and the like. The system 100 may comprise a user device 107, which may comprise a graphical user interface 110, a risk assessment engine 115, and a database 120. The system 100 may also comprise a communication network 125, a server 130, and a display 135.

According to various embodiments, the graphical user interface 110 may be configured to receive user data from the user 105. The graphical user interface 110 may comprise any suitable system for communicating, accessing, updating, exchanging, organizing, and/or managing information such as by data collection, encryption, acquisition, storage, dissemination, and the like. In one embodiment, the graphical user interface 110 may comprise a mobile device interface. In another embodiment, the graphical user interface 110 may comprise a website interface.

According to various embodiments, the user 105 may access the graphical user interface 110 by entering an email address, password, and/or pin into the graphical user interface 110. Once the user 105 has access to the graphical user interface 110, the user 105 may enter the user data, such as identifying information about the user 105, field arrest information about the victim's abuser, and the like, into the graphical user interface 110. The identifying information may comprise any suitable information associated with the identity of the user 105. For example, the identifying information may comprise the victim's first name, last name, and relationship to the abuser. The identifying information may also comprise an incident number and a computer aided dispatch (CAD). Similarly, the field arrest information may comprise any suitable information about the suspect. For example, the field arrest information may comprise the suspect's photo, name, alias, date of birth, ethnicity, gender, race, transient status, home address, and immigration status.

According to various embodiments, the field arrest information may be used to create a profile of the abuser, and the identifying information may be used to create a profile of the victim. Both the victim's profile and the abuser's profile may be saved to the database 120. If the identifying information changes at a later time, the victim's profile may be updated and saved to the database 120 to reflect the changes. Similarly, if the field arrest information changes at a later time, the abuser's profile may be updated and saved to the database 120 to reflect the changes.

The risk assessment engine 115 may be communicatively linked to the graphical user interface 110 and may be configured to provide a series of assessment questions to the user 105 via the graphical user interface 110. The user 105 may be asked to respond with simple “yes” or “no” answers. Alternatively, the user 105 may decline to respond. The assessment questions may be formulated according to any suitable risk assessment tool, such as a standardized harm assessment risk tool (i.e., H.A.R.T.), and the like, to ascertain whether the victim is at a high-risk of death or serious injury. In the case of domestic violence, some risk factors that may indicate whether a victim in an abusive relationship may be at a high-risk of death or serious injury may include past death threats made by the abuser, the abuser's employment status, and the abuser's access to a gun. For example, the following assessment questions may be provided to the user 105 via the graphical user interface 110:

Q1. Has your (former) partner ever used a weapon against you or threatened you with a weapon?
Q2. Has your (former) partner threatened to kill you or your children?
Q3. Do you think your (former) partner might try to kill you?
Q4. Does your (former) partner have a gun or can they get one easily?
Q5. Has your (former) partner ever tried to strangle (choke) you?
Q6. Is your (former) partner violently or constantly jealous, or do they control most of your daily activities?
Q7. Have you left your (former) partner or separated after living together or being married?
Q8. Is your (former) partner unemployed?
Q9. Has your (former) partner ever tried to kill themselves?
Q10. Do you have a child that your (former) partner knows is not theirs?
Q11. Does your (former) partner follow or spy on you or leave threatening messages?

It will be appreciated that the assessment questions may include, but are not limited to, the assessment questions discussed above. After the victim has finished answering the assessment questions, the risk assessment engine 115 may retrieve the identifying information and the answers and analyze the assessment information and the answers. The risk assessment engine 115 may be configured to automatically or upon initiation by the user 105 perform processing of the answers and the identifying information to produce a risk profile of the user 105.

The risk profile may comprise a risk score which may be a low-risk score, a moderate risk score, or a high-risk score. The computed risk score may be compared to a reference score, where the reference score may depend on the particular harm assessment risk tool that is used. The higher the risk score, the more likely the victim is to experience death or serious injury during a disagreement or fight with the abuser. In contrast, the lower the score, the less likely the victim is to experience death or serious injury during a disagreement or fight with the abuser. For instance, a low-risk score may indicate that the victim may be more likely to experience verbal or symbolic aggression than physical aggression during a disagreement or fight with the abuser. A medium risk score may indicate that the victim may be likely to experience physical aggression with a low to moderate risk of death or serious injury during a disagreement or fight with the abuser. A high-risk score may indicate that the victim may be likely to experience physical aggression with a high-risk of death or serious injury during a disagreement or fight with the abuser.

The risk score may be a weighted score. Specifically, the risk score may be computed by attributing one or more points to each “yes” answer, while attributing no points to each “no” answer. The number of points attributed to a particular answer may depend on its corresponding question. For example, some “yes” answers may be given more weight than other “yes” answers. The points may be summed to compute the risk score.

In some embodiments, the risk assessment engine 115 may assign a plurality of number values to a plurality of responses received from the user 105. Each response may be assigned a number value comprising at least one of a first number value or a second number value. It will be appreciated that each of the first number value and the second number value may comprise any suitable number value, such as 1, 2, 3, and the like, and may depend on the assessment question and/or answer choices. In addition, the risk assessment engine 115 may assign a respective one of a plurality of weighted values to each number value. The weighted values may comprise any suitable fraction, ratio, or percentage. Furthermore, the weighted values may be predetermined.

It will be appreciated that modifications may be made to the system 100 without departing from the scope of the present invention. For example, in one embodiment, the system 100 may further comprise an interface (not shown). The interface (not shown) may be a dynamic interface and may be communicatively linked to the display 135. The interface (not shown) may be configured in any suitable such that the user 105 may develop and/or customize the assessment questions depending on the victim's situation. For instance, the assessment questions may be selected according to the victim. It will also be appreciated that the system 100 may recommend one or more “community partners” to the user 105. Specifically, the system 100 may recommend the one or more “community partners” to the user 105 based on the user data and the geographical location of the user. For example, the “community partners” may include, but are not limited to, homeless shelters, substance abuse rehabilitation centers, and mental health clinics that are best suited to assist the victim in developing a safety plan.

In addition, the display 135 may present the one or more “community partners” to the user 105 in an improved manner, such as by displaying the most relevant “community partners” at the top of the display 135 and the less relevant “community partners” at the bottom of the display 135. Further, the display 135 may present a variety of information about the one or more “community partners,” such as the type of services the one or more “community partners” offer, and the like. For instance, the one or more community partners may comprise a homeless shelter, a substance abuse rehabilitation center, a mental health clinic, a domestic violence center, and the like. It will be further appreciated that, in some embodiments, the system 100 may alert and/or notify the one or more “community partners” of a referral and/or an arrest that was made.

The user data may comprise “Criminal Offender Record Information” (CORI), which may comprise confidential data and/or information, i.e., data and/or information that is desired to be kept confidential. According to various embodiments, the system 100 may automatically redact the CORI from the user data prior to sending the user data to the one or more “community partners”. Specifically, the system 100 may be configured to disseminate the CORI data to the relevant stakeholders according to memorandums of understanding (MOUs), i.e., agreements between two or more of the stakeholders.

The system 100 may transmit the user data to the relevant stakeholders, including, but not limited, to the one or more “community partners” so that the relevant stakeholders may utilize the user data to optimize human resource allocation, training, and/or budget allocation. For example, in one embodiment, the display 135 may present the user data in one or more bar charts. In an alternative embodiment, the display 135 may present the user data, regardless of where the user data was collected, in a variety of chart formats (other than bar charts), where such charts may be exported into a Microsoft Excel document or a PDF document.

According to various embodiments, the database 120 may be accessible to the risk assessment engine 115. In particular, the database 120 may be communicatively linked to the risk assessment engine 115. The database 120 may be configured to securely store the identifying information, field arrest information, and risk profile until the information is transmitted from the database 120 to the server 130 over the communication network 125.

According to various embodiments, the system may be connected by the communication network 125. The communication network 125 may be a private network and may be secure. The communication network 125 may comprise any suitable system for exchanging data, such as the Internet, off-line communications, wireless communications, and/or the like.

It will be appreciated that for security reasons, any databases, systems, and/or components of the present technology may comprise any combination of databases or components at a single location or at multiple locations, wherein each database or system includes any of various suitable security features, such as firewalls, access codes, encryption, de-encryption, compression, decompression, and/or the like.

According to various embodiments, the server 130 may be communicatively linked to the database 120 over the communication network 125, such that the server 130 may be accessed by one or more law enforcement agencies and/or victim advocacy groups. The server 130 may be configured to receive the identifying information, field arrest information, and risk profile from the database 120. Once the identifying information, field arrest information, and risk profile are received by the server 130, the identifying information, field arrest information, and risk profile may be accessed through any suitable user profile module. The user profile module may be accessible via any suitable web portal, such as “VDQ.studio”, and may be presented on the display 135. In some embodiments, the server 130 may be provided with selective access to the identifying information, field arrest information, and risk profile such that the identifying information, field arrest information, and risk profile may only be accessed by one or more designated system administrators.

In some embodiments, the database 120 may comprise a database encryption module 121 and the server 130 may comprise a server encryption module 131. The database encryption module 121 and the server encryption module 131 may be configured to encrypt the communication link between the database 120 and the server 130. In particular, the encryption modules 121, 131 may encrypt the messages and/or information that is exchanged between the database 120 and the server 130. The encryption modules 121, 131 may be turned on or off at the discretion of the user and may be configured to encrypt the messages in any suitable manner, such as by key cryptography, hash functions, and the like.

Each system administrator may manage data associated with its respective law enforcement agency, victim advocacy group, and the like (i.e., organizational unit or “O-unit”). Each system administrator may perform a variety of operations through the web portal. For example, each system administrator may access the user profile module and any other module of the system 100 through the web portal. Each system administrator may create and manage different user accounts within the system 100 in the user profile module. Each system administrator may also analyze the incidents reported, view assessments and field arrests, manage alerts, manage distribution groups and teams and view audit logs.

Each system administrator may share data associated with its respective O-unit with other O-units. For example, each system administrator may create a new O-unit in the user profile module and then provide the new O-unit with access to the web portal. To create a new O-unit, each system administrator may click the “+New O-Unit” button and provide the name of the specific O-unit, a description of the O-unit, and a 9-character Originating Agency Identification (ORI) assigned to the O-unit. Once the O-unit has been created, each system administrator may then create a new O-unit administrator. Each system administrator may create the new O-unit administrator by clicking on the “New O-Unit Admin” button and providing the name and email address of the specific O-unit administrator. Once the O-unit administrator has been granted access to the web portal, the identifying information, field arrest information, and risk profile may be shared with the newly added O-unit administrator.

Each system administrator may select the specific data to be shared with the selected O-unit from a dropdown menu. Under the “Field Arrest” heading, each system administrator may select “Full” to share an unredacted version of the field arrest information with the O-unit, or “None” to not share any of the field arrest information with the O-unit. Similarly, under the “Assessment” heading, each system administrator may select “None”, “Full”, “Redacted”, or “High Risk Only”. “None” may be selected to not share any of the data with the O-unit; “Full” may be selected to share all the data with the O-unit; “Redacted” may be selected to share the data with the O-unit but with certain portions of it hidden; and “High Risk Only” may be selected to share only the high-risk data with the O-unit.

It will be appreciated that each system administrator may share data associated with its respective O-unit with other O-units in any suitable manner. In some embodiments, each system administrator may select the “Submit” button to provide the selected O-unit with a weblink to access the web portal. In other embodiments, such as where the selected O-unit has not been granted access to the web portal, each system administrator may send the selected O-unit unit a secure message, such as an encrypted email, to access the data.

In operation, when a domestic violence, transient, mental health, and substance abuse incident is reported to a law enforcement agency, such as a local police department, county sheriff, and the like, the law enforcement agency may respond by sending one or more police officers or victim advocates to the location where the incident occurred. Once on scene, the police officers, or any frontline first responder may collect critical domestic violence, transient, mental health, and substance abuse information and then connect the victim/respondent with the government agency and/or victim advocacy groups that are best suited to assist the victim/respondent.

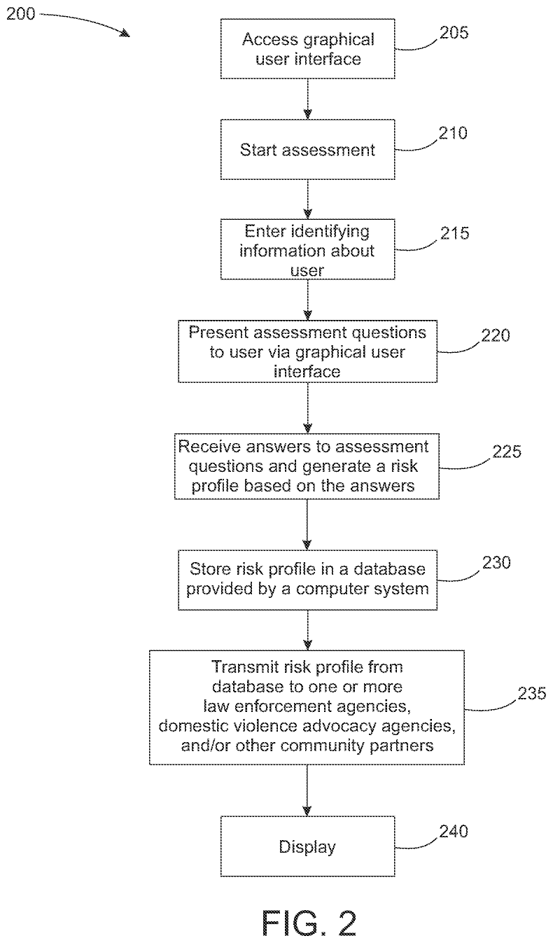

Referring to FIG. 2, a first method of operation 200 according to various aspects of the present technology may comprise accessing the graphical user interface 110 by the user 105 (205). The user 105 may log into its user account via the mobile application by entering its email and/or password into the graphical user interface 110. Once the user 105 is logged into its user account, the first method of operation 200 may also comprise starting an assessment by clicking on the “Start Assessment” button (210). In addition, the first method of operation 200 may comprise entering identifying information about the user 105 into the graphical user interface 110 (215). For example, in the case of a domestic violence incident, the police officer may provide the victim's first name, last name, and relationship to the suspect along with the incident number and the computer aided dispatch (CAD) number. The police officer may then press the “Continue” button. After pressing the “Continue” button, the police officer may hand over the mobile device to the victim. At this point, the system 100 may present the assessment questions to the victim via the graphical user interface 110 (220). The victim may be asked to respond with simple “yes” or “no” answers. Alternatively, the victim may decline to respond.

After the victim has finished answering the questions, the risk assessment engine 115 may retrieve the answers and the identifying information about the victim and analyze both the answers and the identifying information and perform any suitable processing of the answers and the identifying information to generate the victim's risk profile (225). As is described at paragraphs [0016] and [0017] in the instant application, the risk assessment engine 115 may evaluate the answers to determine and calculate an appropriate risk score for the user 105. The risk profile may comprise the risk score, and the risk score may indicate how likely the victim is to experience death or serious injury during a disagreement or fight with the abuser. The first method of operation 200 may further comprise storing the risk profile in the database 120 (230). At any time, the identifying information and risk profile may be transmitted from the database 120 to the server 130 of a law enforcement agency and/or a victim advocacy group that is best suited to assist the victim in developing a safety plan (235). Once the risk profile is received by the server 130, the risk profile may be displayed via the display 135 (240).

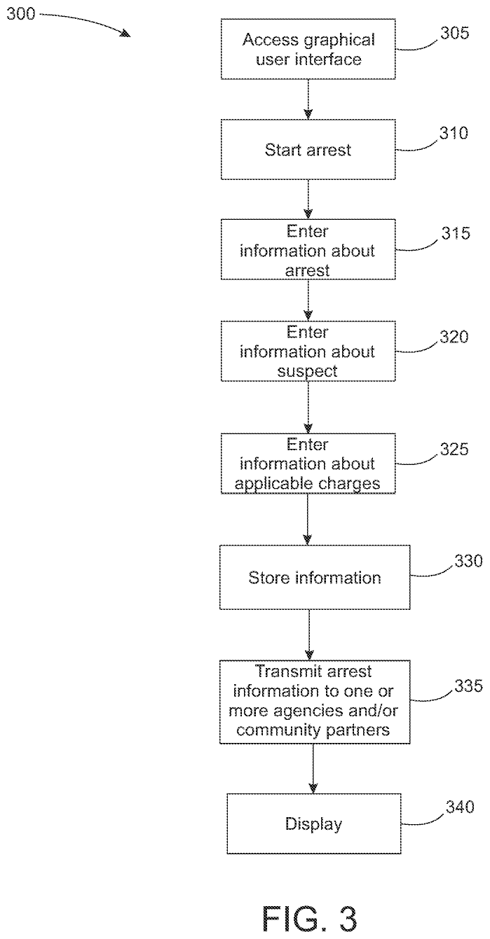

Referring now to FIG. 3, a second method of operation 300 according to various aspects of the present technology may comprise accessing the graphical user interface 110 by the user 105 (305). The user 105 may log into its user account via the mobile application by entering its email and/or password into the graphical user interface 110. Once the user 105 is logged into its user account, the second method of operation 300 may also comprise starting an arrest by clicking on the “Start Arrest” button (310). In addition, the second method of operation 300 may comprise entering information about the arrest into the graphical user interface 110 (315). For example, in the case of a domestic violence incident, the police officer may provide the incident number, computer aided dispatch (CAD) number, case number, date and time of the arrest, location of the arrest along with the arresting officer's name, badge number, agency name, and email. In addition, the police officer may provide any additional notes. For example, the police officer may indicate whether the suspect resisted arrest and/or whether the police officer used force at the time of the arrest. The police officer may then click the “Save and Next” button.

After pressing the “Save and Next” button, the second method of operation 300 may further comprise entering information about the suspect into the graphical user interface 110 (320). For example, the police officer may provide the suspect's photo, name, alias, date of birth, ethnicity, gender, race, transient status, home address, immigration status, and the like. In addition, the police officer may provide additional notes. For example, the police officer may indicate whether fingerprints of the suspect have been identified in police records and whether a warrant check was done. Further, the police officer may provide the State Identification Index (SII) number of the suspect, if the suspect has one, the driver's license number of the suspect, the Federal Bureau of Investigation (FBI) number of the suspect, if the suspect has an FBI number attached to him or her, and any other arrest notes. The police officer may then click the “Save and Next” button.

After pressing the “Save and Next” button, the second method of operation 300 may further comprise entering information about applicable charges into the graphical user interface 110 (325). The second method of operation 300 may further comprise storing information related to the arrest, suspect, and applicable charges (collectively, the “field arrest information”) in the database 120 (330). At any time, the field arrest information may be transmitted from the database 120 to the server 130 of a law enforcement agency and/or a victim advocacy group that is best suited to assist the victim in developing a safety plan (335). Once the risk profile is received by the server 130, the risk profile may be displayed via the display 135 (340).

The particular implementations shown and described are illustrative of the technology and its best mode and are not intended to otherwise limit the scope of the present technology in any way. Indeed, for the sake of brevity, conventional manufacturing, connection, preparation, and other functional aspects of the apparatus may not be described in detail. Furthermore, the connections and points of contact shown in the various figures are intended to represent exemplary physical relationships between the various elements. Many alternative or additional functional relationships or physical connections may be present in a practical system.

Embodiments of the present technology may be used in conjunction with any number of methods and devices for optimizing a risk assessment process. In the foregoing description, the technology has been described with reference to specific exemplary embodiments. Various modifications and changes may be made, however, without departing from the scope of the present technology as set forth. The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the steps recited in any method or process embodiment may be executed in any appropriate order and are not limited to the explicit order presented in the specific examples. Additionally, the components and/or elements recited in any system embodiment may be combined in a variety of permutations to produce substantially the same result as the present technology and are accordingly not limited to the specific configuration recited in the specific examples.

Benefits, other advantages, and solutions to problems have been described above with regard to particular embodiments. Any benefit, advantage, solution to problems or any element that may cause any particular benefit, advantage, or solution to occur or to become more pronounced, however, is not to be construed as a critical, required, or essential feature or component.

The terms “comprises,” “comprising,” or any variation thereof, are intended to reference a non-exclusive inclusion, such that a process, method, article, composition, or apparatus that comprises a list of elements does not include only those elements recited but may also include other elements not expressly listed or inherent to such process, method, article, composition, or apparatus. Other combinations and/or modifications of the above-described structures, arrangements, applications, proportions, elements, materials, or components used in the practice of the present technology, in addition to those not specifically recited, may be varied, or otherwise particularly adapted to specific environments, manufacturing specifications, design parameters or other operating requirements without departing from the general principles of the same.

The present technology has been described above with reference to an exemplary embodiment. However, changes and modifications may be made to the exemplary embodiment without departing from the scope of the present technology. These and other changes or modifications are intended to be included within the scope of the present technology.

1. A system for optimizing a risk assessment process, comprising:
a user device, comprising:
a graphical user interface configured to receive user data, wherein the user data comprises identifying information;
a risk assessment engine configured to compute a risk score and generate a risk profile utilizing the identifying information and the risk score, wherein the risk profile indicates whether a user is likely to be harmed; and
a database configured to securely store the risk profile;
a server communicatively linked to the database over a communication network, wherein the server is provided with selective access to the risk profile; and
a display configured to present the risk profile.

2. The system of claim 1, wherein the user is at least one of a victim of domestic violence, an individual experiencing homelessness, an individual experiencing mental health issues, or an individual experiencing substance abuse issues.

3. The system of claim 1, wherein the display is further configured to present a community partner to the user based on the computed risk score and a geographical location of the user, and wherein the community partner comprises at least one of a homeless shelter, substance abuse rehabilitation center, mental health clinic, or domestic violence center.

4. The system of claim 1, wherein each of the database and the server comprises an encryption module configured to encrypt the communication link between the database and the server.

5. The system of claim 1, wherein:
the graphical user interface is further configured to:
present a plurality of assessment questions to the user via the graphical user interface; and
receive a plurality of responses from the user, wherein the user is prompted to select between a first answer and a second answer for each response; and
the risk assessment engine is further configured to:
assign a plurality of number values to the plurality of responses, wherein each response is assigned a number value comprising at least one of a first number value or a second number value; and
compute the risk score according to the plurality of number values.

6. The system of claim 5, wherein the plurality of assessment questions are formulated according to a harm assessment risk tool.

7. The system of claim 5, wherein the computed risk score comprises at least one of a low-risk score relative to a reference risk score, a medium-risk score relative to the reference risk score, or a high-risk score relative to the reference score, and wherein:
the low-risk score indicates a low probability that the user will be harmed;
the medium risk score indicates a medium probability that the user will be harmed; and
the high risk score indicates a high probability that the user will be harmed.

8. The system of claim 5, wherein the risk score is equal to a weighted average sum of the plurality of number values.

9. The system of claim 8, wherein each number value is assigned a respective one of a plurality of weighted values, and wherein the plurality of weighted values are predetermined.

10. A method for optimizing a risk assessment process, comprising:
presenting, via a graphical user interface, a plurality of assessment questions to a user;
receiving, via the graphical user interface, a plurality of responses from the user, wherein the user is prompted to select between a first answer and a second answer for each response;
assigning, via a risk assessment engine, a plurality of number values to the plurality of responses, wherein each response is assigned a respective one of the plurality of number values;
utilizing the risk assessment engine to compute a risk score according to the plurality of number values; and
generating, via the risk assessment engine, a risk profile utilizing the risk score and the identifying information.

11. The method of claim 10, wherein the user is at least one of a victim of domestic violence, an individual experiencing homelessness, an individual experiencing mental health issues, or an individual experiencing substance abuse issues.

12. The method of claim 10, further comprising presenting, via a display, to the user a community partner based on the computed risk score and a geographical location of the user, wherein the community partner comprises at least one of a homeless shelter, substance abuse rehabilitation center, mental health clinic, or domestic violence center.

13. The method of claim 10, wherein the plurality of assessment questions are formulated according to a harm assessment risk tool.

14. The method of claim 10, wherein the computed risk score comprises at least one of a low-risk score relative to a reference risk score, a medium-risk score relative to the reference risk score, or a high-risk score relative to the reference score, and wherein:
the low-risk score indicates a low probability that the user will be harmed;
the medium-risk score indicates a medium probability that the user will be harmed; and
the high-risk score indicates a high probability that the user will be harmed.

15. The method of claim 10, wherein the risk score is equal to a weighted average sum of the plurality of number values.

16. The method of claim 15, wherein each number value is assigned a respective one of a plurality of weighted values, and wherein the plurality of weighted values are predetermined.
